(19) United States
(12) Patent Application Publication
(10) Pub. No.: US 2005/0186296 A1
(43) Pub. Date: Aug. 25, 2005

(54) Profiles of lipid proteins and inhibiting HMG-CoA reductase

(76) Inventors: Afa Kehaati Palu, Orem, UT (US); Claude Jarakae Jensen, Cedar Hills, UT (US); Mian Ying Wang, Rockford, IL (US); Bing-Nan Zhou, Pleasant Grove, UT (US); Stephen Story, Alpine, UT (US)

Correspondence Address: KIRTON AND MCCONKIE;1800 EAGLE GATE TOWER, 60 EAST SOUTH TEMPLE, P O BOX 45120, SALT LAKE CITY, UT, 84145-0120, US

(21) Appl. No.: 11/034,505

(22) Filed: Jan. 13, 2005

Related U.S. Application Data
(60) Provisional application No. 60/536,663, filed on Jan. 15, 2004.
(60) Provisional application No. 60/552,144, filed on Mar. 10, 2004.

(51) Int. Cl. A61K 35/78

(52) U.S. Cl. 424/769

(57) ABSTRACT

The present invention comprises methods and compositions for improving profiles of lipid proteins, reducing low-density lipoprotein levels, increasing high-density lipoprotein levels, decreasing the absorption of fatty acids across the intestinal epithelium and inhibiting HMG-CoA Reductase in living organisms utilizing Morinda citrifolia L activity in mammals, including humans.

### RELATED APPLICATIONS

This application claims priority to U.S. Patent Application Ser. No. 60/536,663 filed Jan. 15, 2004, entitled “A Method for Improving Lipoprotein Profiles and Reducing LDL Cholesterol,” and to U.S. Patent Application Ser. No. 60/552,144 filed Mar. 10, 2004, entitled “Methods and Compositions for Inhibiting HMG-CoA Reductase.”

### BACKGROUND OF THE INVENTION

1. Field of the Invention The present invention relates to methods and compositions for improving profiles of lipid protein, reducing LDL cholesterol, decrease the absorption of fatty acids across the intestinal epithelium and inhibiting HMG-CoA Reductase in living organisms utilizing Morinda citrifolia L.

2. Background and Related Art

Cholesterol is a fatty lipid found in the body tissues and blood plasma of vertebrates. Cholesterol can be found in large concentrations in the brain, spinal cord, and liver. The liver is the most important site of cholesterol biosynthesis, although other sites include the adrenal glands and reproductive organs. The insolubility of cholesterol in water is a factor in the development of atherosclerosis, the pathological deposition of plaques of cholesterol and other lipids on the insides of major blood vessels, a condition associated with coronary artery disease.

Recent research has shown that the relative abundance lipoproteins, to which cholesterol becomes attached may be the real cause of cholesterol buildup in the blood vessels. High-density lipoprotein (HDL) carries cholesterol out of the bloodstream for excretion, while low-density lipoprotein (LDL) carries it back into the system for use by various body cells. Researchers believe that HDL and LDL levels in the bloodstream may be at least as important as cholesterol levels, and now measure both to determine risk for heart disease.

Maintaining a healthy cholesterol level in the blood is crucial to the health of many living organisms, including human beings. Cholesterol synthesis can be effectively blocked by a class of compounds called statins. Statins are potent competitive inhibitors (Kᵢ<1 nM) of HMG-CoA reductase, the essential control point in the biosynthetic pathway. HMG-CoA Reductase is particularly responsible for cholesterol synthesis. Inhibition of HMG-CoA Reductase decreases excess cholesterol production. Plasma cholesterol levels decrease by 50% in many patients given both statin and inhibitors of bile-salt reabsorption. Inhibitors of HMG-CoA reductase are widely used to lower the plasma cholesterol level in people who have atherosclerosis, which is the leading cause of death in industrialized societies.

A study reported in 1998 that HMG-CoA Reductase inhibitors protect against stroke through endothelial nitric oxide synthase. Treatment of ischemic strokes is limited to prophylactic agents that block the coagulation cascade so that no plaque forms inside the arteries. Plaque formations inside arteries reduce arterial volume and restricts blood flow, thereby increasing the blood pressure to abnormal levels. This study showed for the first time that HMG-CoA Reductase inhibitors are cholesterol-lowering agents that protect against cerebral injury by an unidentified mechanism that involves the selective up-regulation of endothelial NO synthase (eNOS). The prophylactic treatment with HMG-CoA Reductase inhibitors augments cerebral blood flow, thus reducing cerebral infarct size and ultimately improves the neurological functions in normocholesterolemic mice. This study concluded that HMG-CoA Reductase inhibitors provide a prophylactic treatment strategy for increasing blood flow and reducing brain injury during cerebral ischemia (localized tissue anemia due to obstruction of the inflow of arterial blood).

Another study reported in 2003 the effects of HMG-CoA Reductase inhibitors on cardiovascular diseases. This study found that HMG-CoA Reductase inhibitors lower the level of circulating LDL cholesterol by blocking the action of HMG-CoA Reductase. In several clinical trials, the following additional benefits were discovered in addition to the cholesterol lowering benefits: improvements in vasoreactivity, homeostasis, plaque stability, reduction of proinflammatory events such as decreases in monocyte adhesion and infiltration. These benefits account for why statins help to treat or prevent cardiovascular diseases.

Many types of statins on the market are designed to inhibit the HMG-CoA Reductase enzyme. Some of the drugs are synthetic, and some are derived from natural sources, such as from fungi. Some examples of the statins or HMG-CoA Reductase inhibitors include: Lovastatin, marketed under the brand name MEVACOR™, Simvastativ, marketed under brand name ZOCOR™, Pravastatin, marketed under the brand name PRAVACHOL™, Fluvastatin, marketed under the brand name LESCOL™, and Atorvastatin, marketed under the brand name LIPITOR™.

As noted above, statins have many beneficial effects for the human body. However, as with many drugs, statins also have various known undesirable side effects. For example, some common side effects of existing statins include: muscle tenderness or soreness, unexplained muscle pain, general malaise, fatigue and weakness, fever, weakness, and flu-like illness. Moreover, statins generally are not recommended for those who have liver diseases, are pregnant or planning to be pregnant, are breast feeding, or who drink more than 1-2 alcoholic drinks per day.

In 2002, it was reported that homocysteine induces the unregulated expression of the HMG-CoA Reductase enzyme, which increases the production of cholesterol in the body. Consequently, homocysteine suppresses the production of nitric oxide. The administration of statin and statin-like drugs reduces cholesterol synthesis and improves endothelial function, thereby restoring cardiovascular health.

Elevated blood cholesterol is one of the major modifiable risk factors for coronary heart disease (CHD), the leading cause of death in the U.S. CHD accounts for approximately 490,000 deaths each year, and angina and nonfatal myocardial infarction (MI) are a source of substantial morbidity. CHD is projected to cost over $60 billion in 1995 in the U.S. in medical expenses and lost productivity. Clinical events are the result of a multifactorial process that begins years before the onset of symptoms. Autopsy studies detected early lesions of atherosclerosis in many adolescents and young adults. The onset of atherosclerosis and symptomatic CHD is earlier among persons with inherited lipid disorders such as familial hypercholesterolemia (FH) and familial combined hyperlipidemia (FCH).

Epidemiologic, patho-logic, animal, genetic, and clinical studies support a causal relationship between blood lipids (usually measured as serum levels) and coronary atherosclerosis. High cholesterol is a risk factor for CHD. Because CHD is a multifactorial process, however, the re is no definition of high cholesterol that discriminates well between individuals who will or will not develop CHD. The risk associated with high total cholesterol is primarily due to high levels of low-density lipoprotein cholesterol (LDL-C), but there is a strong, independent, and inverse association between high-density lipoprotein cholesterol (HDL-C) levels and CHD risk.

It is apparent that there is much interest in methods for inhibiting the HMG-CoA Reductase enzyme. Healthy ways to inhibit excess cholesterol production are clearly valuable to the human population and would be invaluable to the art.

### SUMMARY OF THE INVENTION

The invention comprises methods and compositions for improving profiles of lipoproteins, reducing VLDL and LDL lipoprotein levels, increasing HDL lipoprotein levels, decreasing the absorption of fatty acids across the intestinal epithelium, inhibiting HMG-CoA Reductase and reducing total blood cholesterol in living organisms utilizing Morinda citrifolia L. The invention includes methods and compositions for selectively decreasing LDLs. Embodiments of the present invention comprise methods and compositions for inhibiting HMG-CoA Reductase without causing the negative side effects associated with statins currently available on the market.

The formulations of the invention comprise processed Morinda citrifolia products. In one embodiment, the formulations include one or more extracts from the Morinda citrifolia L. plant. The Morinda citrifolia extracts preferably include Morinda citrifolia fruit juice, which juice is preferably present in an amount capable of maximizing the inhibition of the HMG-CoA Reductase or for improving lipoprotein profiles without causing negative side effects when the composition is administered to a mammal.

Methods of the present invention comprise the administration and/or consumption of processed Morinda citrifolia products in amounts that inhibit HMG-CoA Reductase and/or improve lipoprotein profiles in mammals. Methods of the present invention also include the obtaining of Morinda citrifolia compositions and extracts, including Morinda citrifolia fruit juice and concentrates thereof.

Some embodiments of the invention provide methods of inhibiting the activity of HMG-CoA Reductase without causing the negative secondary effects caused by known statins. Some embodiments of the invention provide an orally administered HMG-CoA Reductase inhibitor capable of use during pregnancy. Some embodiments of the invention provide an orally administered composition capable of inhibiting HMG-CoA Reductase activity in patients that do not respond to known statins. Some embodiments of the invention provide an over-the-counter composition for inhibiting HMG-CoA Reductase activity in mammals without requiring a prescription.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### DETAILED DESCRIPTION OF THE INVENTION

The following description of embodiments of the methods and compositions of the present invention is not intended to limit the scope of the invention, but is merely representative of some embodiments, including the preferred embodiments, of the present invention.

The invention comprises methods and compositions for improving profiles of lipoproteins, reducing VLDL and LDL lipoprotein levels, increasing HDL lipoprotein levels, decreasing the absorption of fatty acids across the intestinal epithelium, inhibiting HMG-CoA Reductase and reducing total blood cholesterol in living organisms utilizing Morinda citrifolia L.

The present invention comprises Morinda citrifolia compositions, each of which include one or more extracts from the Morinda citrifolia L. plant. The Morinda citrifolia extracts preferably include Morinda citrifolia fruit juice, which juice is preferably present in an amount capable of maximizing the inhibition of HMG-CoA Reductase without causing negative side effects when the composition is administered to a mammal. Extracts of the Morinda citrifolia plant may include one more parts of the Morinda citrifolia plant, including but not limited to the: fruit, including the fruit juice and fruit pulp and concentrates thereof, fruit puree, leaves, including leaf extract, seeds, including the seed oil, flowers, roots, bark, and wood.

Some compositions of the present invention comprise Morinda citrifolia extracts present between about 1 and 5 percent of the weight of the total composition. Other such percentage ranges include: about 0.1 and 50 percent; about 85 and 99 percent; about 5 and 10 percent; about 10 and 15 percent; about 15 and 20 percent; about 20 and 50 percent; and about 50 and 100 percent.

In some Morinda citrifolia compositions of the present invention, Morinda citrifolia fruit juice evaporative concentrate is present, the evaporative concentrate having a concentration strength (described further herein) between about 8 and 12 percent.

In some Morinda citrifolia compositions of the present invention, Morinda citrifolia fruit juice freeze concentrate is present, the freeze concentrate having a concentration strength (described further herein) between about 8 and 12 percent. Other such percentage ranges include: about 4 and 12 percent; and about 0.5 and 12 percent.

One or more Morinda citrifolia extracts can be further combined with other ingredients or carriers (discussed further herein) to produce a pharmaceutical Morinda citrifolia product or composition (“pharmaceutical” herein referring to any drug or product designed to improve the health of living organisms such as human beings or mammals, including nutraceutical products) that is also a Morinda citrifolia of the present invention. Examples of pharmaceutical Morinda citrifolia products may include, but are not limited to, orally administered solutions and intravenous solutions.

Methods of the present invention comprise the administration and/or consumption of Morinda citrifolia compositions in amounts that improve profiles of lipoproteins, reduce LDL cholesterol, decrease the absorption of fatty acids across the intestinal epithelium and inhibit HMG-CoA Reductase in living organisms utilizing Morinda citrifolia L activity in mammals, including humans. It will be understood that specific dosage levels of any compositions that will be administered to any particular patient will depend upon a variety of factors, including the patient's age, body weight, general health, gender, diet, time of administration, route of administration, rate of excretion, drug combination, and the severity of the particular diseases undergoing therapy or in the process of incubation.

Methods of the present invention also include the obtaining of Morinda citrifolia compositions and extracts, including Morinda citrifolia fruit juice and concentrates thereof. It will be noted that some of the embodiments of the present invention contemplate obtaining the Morinda citrifolia fruit juice pre-made. Various methods of the present invention shall be described in more detail further herein.

The following disclosure of the present invention is grouped into subheadings, The utilization of the subheadings is for convenience of the reader only and is not to be construed as limiting in any sense.

1. Obtaining Extracts From Morinda citrifolia Plant for Incorporation Into the Compositions of the Present Invention

The Indian Mulberry or Noni plant, known scientifically as Morinda citrifolia L. (Morinda citrifolia), is a shrub or small tree. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or round, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Morinda citrifolia plant was as a red and yellow dye source. Recently, there has been an interest in the nutritional 10 and health benefits of the Morinda citrifolia plant, further discussed below.

Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in other products. In some embodiments, the juice and pulp can be pureed into a homogenous blend to be mixed with other ingredients. Other processes include freeze-drying the fruit and juice. The fruit and juice can be reconstituted during production of the final juice product. Still other processes include air-drying the fruit and juices, prior to being masticated.

The present invention also contemplates the use of fruit juice and/or puree fruit juice extracted from the Morinda citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing, occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit may be placed in containers for processing and transport. In a preferred embodiment of the invention, the aged fruit is placed in plastic lined containers for further processing and transport. The containers of aged fruit may be held from 0 to 120 days. In a preferred embodiment of the invention, the fruit containers are held for 7 to 14 days before processing. The containers can optionally be stored under refrigerated conditions or ambient/room temperature conditions prior to further processing. The fruit is unpacked from the storage containers and may be further processed through a manual or mechanical separator, in which the seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions.

The Morinda citrifolia juice and pulp are preferably blended in a homogenous blend, after which they may be mixed with other ingredients. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp separated from the seeds and is different from the fruit juice product described herein.

Each product is filled and sealed into a final container. The container may be plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 0.01 micron up to 2000 microns, more preferably less than 500 microns, a filter press, reverse osmosis filtration, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp may be washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

The processed Morinda citrifolia product may also exist as a fiber. Still further, the processed Morinda citrifolia product may also exist in oil form, such as an oil extract. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The high fiber product may include wet or dry Morinda citrifolia pulp, supplemental fiber ingredients, water, sweeteners, flavoring agents, coloring agents, and/or nutritional ingredients. The supplemental fiber ingredients may include plant based fiber products, either commercially available or developed privately. Examples of some typical fiber products are guar gum, gum arabic, soybean fiber, oat fiber, pea fiber, fig fiber, citrus pulp sacs, hydroxymethylcellulose, cellulose, seaweed, food grade lumber or wood pulp, hemicellulose, etc. Other supplemental fiber ingredients may be derived from grains or grain products. The concentrations of these other fiber raw materials typically range from 0 up to 30 percent, by weight, and more preferably from 10 to 30 percent by weight.

The juice and pulp can be dried using a variety of methods. The juice and pulp mixture can be pasteurized or enzymatically treated prior to drying. The enzymatic process begins with heating the product to a temperature between 75° F. and 135° F. It is then treated with either a single enzyme or a combination of enzymes. These enzymes include, but are not limited to, amylase, lipase, protease, cellulase, bromelin, etc. The juice and pulp may also be dried with other ingredients, such as those described above in connection with the high fiber product. The typical nutritional profile of the dried juice and pulp is 1 to 20 percent moisture, 0.1 to 15 percent protein, 0.1 to 20 percent fiber, and the vitamin and mineral content.

The filtered juice and the water from washing the wet pulp are preferably mixed together. The filtered juice may be vacuum evaporated to a brix of 40 to 70 and a moisture of 0.1 to 80 percent, more preferably from 25 to 75 percent. The resulting concentrated Morinda citrifolia juice may or may not be pasteurized. For example, the juice would not be pasteurized in circumstances where the sugar content or water activity was sufficiently low enough to prevent microbial growth.

The Morinda citrifolia plant is rich in natural ingredients. Those ingredients that have been discovered include: (from the leaves): alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; (from the flowers): acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamosyl-glucopyranoside; (from the fruit): acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; (from the roots): anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; (from the root bark): alizarin, chlororubin, glycosides (pentose, hexose), morindadiol, morindanigrine, morindine, morindone, resinous matter, rubiadin monomethyl ether, and soranjidiol; (from the wood): anthragallol-2,3-dimethylether; (from the tissue culture): damnacanthal, lucidin, lucidin-3-primeveroside, and morindone-6beta-primeveroside; (from the plant): alizarin, alizarin-alpha-methyl ether, anthraquinones, asperuloside, hexanoic acid, morindadiol, morindone, morindogenin, octanoic acid, and ursolic acid.

The present invention contemplates utilizing all parts of the M. citrifolia plant alone, in combination with each other or in combination with other ingredients. The above listed portions of the M. citrifolia plant are not an exhaustive list of parts of the plant to be used but are merely exemplary. Thus, while some of the parts of the M. citrifolia plant are not mentioned above (e.g., seed from the fruit, the pericarp of the fruit, the bark or the plant) the present invention contemplates the use of all of the parts of the plant.

Ingredients, components or extracts may be obtained from any part of the Morinda citrifolia plant including leaves, stem, seeds and/or roots. In a preferred embodiment of the invention, extracts may be obtained from the leaves, stem, seeds, and/or roots by first chopping the raw material. Next, an extraction method may be utilized to isolate ingredients of interest. Extraction of ingredients of interest may be accomplished by exposing the raw ingredients to a solvent of choice. In one embodiment of the invention, a hot water extraction method is utilized, at an appropriate temperature to ensure isolation of the desired ingredients. For example, water may be added to the raw materials in a five to one ratio by weight and heated to 95° C. Other solvents may be utilized for the extraction including organic solvents or mixtures of aqueous and organic solvents. Organic solvents are preferably selected from a list comprising ethanol, methanol, and hexane. Moreover, wet pressure and heat process using ordinary autoclave equipment may be applied. Furthermore, treatment processes using cellulose hydrolysis enzyme may be added to aforementioned processes. After removing insoluble components through filtering, if desired, from extract obtained from leaves, stems, seeds and/or roots, solvent is removed and extract of the present invention is obtained. This extract may be pasteurized, if necessary, or concentrated or dried. Drying may be achieved using ordinary spray drying or freeze-drying. The extract may be stored under cooling or freezing conditions.

Moreover, oil may be extracted from seeds. Oil may be obtained by drying, crushing, and squeezing seeds with a press. More oil may be extracted from seed cake residue by extracting the oil utilizing a solvent selected from a list comprising hexane, ethanol, water, other aqueous solvents, or other organic solvent. The oil contains fatty acid such as linoleic acid, oleic acid, palmitic acid and stearic acid in the form of triglycerides.

Recently, as mentioned, many health benefits have been discovered stemming from the use of products containing Morinda citrifolia. One benefit of Morinda citrifolia is found in its ability to isolate and produce Xeronine. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase, which releases Xeronine from Proxeronine. A paper entitled, “The Pharmacologically Active Ingredient of Noni” by R. M. Heinicke of the University of Hawaii, indicates that Morinda citrifolin is “the best raw material to use for the isolation of xeronine,” because of the building blocks of Proxeronine and Proxeronase.

Xeronine protects and keeps the shape and suppleness of protein molecules so that they may be able to pass through the cell walls and be used to form healthy tissue. Without these nutrients going into the cell, the cell cannot perform its job efficiently. Xeronine assists in enlarging the membrane pores of the cells. This enlargement allows for larger chains of peptides (amino acids or proteins) to be admitted into the cell. If these chains are not used, they become waste. Additionally, Xeronine, which is made from Proxeronine, assists in enlarging the pores to allow better absorption of nutrients. Because of its many benefits, Morinda citrifolia has been known to provide a number of anecdotal effects

As used herein, the term Morinda citrifolia juice refers to a product that includes juice processed from the fruit of the Indian Mulberry or Morinda citrifolia L. plant. In one embodiment, Morinda citrifolia juice includes reconstituted fruit juice from pure juice puree of French Polynesia. The composition or formulation comprising at least one processed Morinda citrifolia product may also include other ingredients. In a further embodiment, Morinda citrifolia juice is not processed from dried or powdered Morinda citrifolia.

2. Formulations and Methods of Administration

The compositions of the present invention may be formulated into any of a variety of compositions, including orally administered compositions, intravenous solutions, and other products or compositions. As mentioned earlier herein, the compositions can include a variety of ingredients.

Orally administered compositions may take the form of, for example, liquids, beverages, tablets, lozenges, aqueous or oily suspensions, dispersible powders or granules, emulsions, syrups, or elixirs. Compositions intended for oral use may be prepared according to any method known in the art, and such compositions may contain one or more agents such as sweetening agents, flavoring agents, coloring agents, and preserving agents. They may also contain one or more additional ingredients such as vitamins and minerals, etc. Tablets may be manufactured to contain one or more Morinda citrifolia extracts in admixture with non-toxic, pharmaceutically acceptable excipients that are suitable for the manufacture of tablets. These excipients may be, for example, inert diluents, granulating and disintegrating agents, binding agents, and lubricating agents. The tablets may be uncoated or they may be coated by known techniques to delay disintegration and absorption in the gastrointestinal tract and thereby provide sustained action over a longer period. For example, a time delay material such as glyceryl monostearate or glyceryl distearate may be used.

Aqueous suspensions may be manufactured to contain Morinda citrifolia extracts in admixture with excipients suitable for the manufacture of aqueous suspensions. Examples of such excipients include, but are not limited to: suspending agents such as sodium carboxymethyl-cellulose, methylcellulose, hydroxy-propylmethycellulose, sodium alginate, polyvinyl-pyrrolidone, gum tragacanth and gum acacia; dispersing or wetting agents such as a naturally-occurring phosphatide like lecithin, or condensation products of an alkylene oxide with fatty acids such as polyoxyethylene stearate, or condensation products of ethylene oxide with long chain aliphatic alcohols such as heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides such as polyethylene sorbitan monooleate.

Typical sweeteners may include, but are not limited to, natural sugars derived from corn, sugar beet, sugar cane, potato, tapioca, or other starch-containing sources that can be chemically or enzymatically converted to crystalline chunks, powders, and/or syrups. In addition, sweeteners can consist of artificial or high intensity sweeteners, some of which are aspartame, sucralose, stevia, saccharin, etc. The concentration of sweeteners may be between from 0 to 50 percent by weight, of the formula, and more preferably between about 1 and 5 percent by weight.

Typical flavors can include, but are not limited to, artificial and/or natural flavor or ingredients that contribute to palatability. Natural flavors include but are not limited to other fruits and vegetables. The concentration of flavors may range, for example, from 0 up to 15 percent by weight, of the formula. Colors may include food grade artificial or natural coloring agents having a concentration ranging from 0 up to 10 percent by weight, of the formula.

Typical nutritional ingredients may include vitamins, minerals, trace elements, herbs, botanical extracts, bioactive chemicals and compounds at concentrations from 0 up to 10 percent by weight. Examples of vitamins one can add to the fiber composition include, but are not limited to, vitamins A, B1 through B12, C, D, E, Folic Acid, Pantothenic Acid, Biotin, etc. Examples of minerals and trace elements one can add to the fiber composition include, but are not limited to, calcium, chromium, copper, cobalt, boron, magnesium, iron, selenium, manganese, molybdenum, potassium, iodine, zinc, phosphorus, etc. Herbs and botanical extracts include, but are not limited to, alfalfa grass, bee pollen, chlorella powder, Dong Quai powder, Ecchinacea root, Gingko Biloba extract, Horsetail herb, Indian mulberry, Shitake mushroom, spirulina seaweed, grape seed extract, etc. Typical bioactive chemicals may include, but are not limited to, caffeine, ephedrine, L-carnitine, creatine, lycopene, etc.

Ingredients of the present invention may also include one or more carrier agents (for example, water) known or used in the art. Examples of other ingredients may include, but are not limited to artificial flavoring, other natural juices or juice concentrates such as a natural grape juice concentrate or a natural blueberry juice concentrate. The ingredients to be utilized in the compositions of the present invention may include any that are safe for internalizing into the body of a mammal.

Favorably, this invention provides a method of diabetes with a Morinda citrifolia-based formulation without any significant tendency to cause undesirable side effects.

The present invention features a unique formulation and method of administering the same to treat affect cholesterol levels, lower LDL cholesterol levels and to inhibit HMG-CoA Reductase, by providing a nutraceutical composition or treatment formulated with one or more processed Morinda citrifolia products derived from the Indian Mulberry plant. The Morinda citrifolia product is incorporated into various carriers or nutraceutical compositions suitable for in vivo treatment of a patient. For instance, the nutraceutical formulation may be ingested orally, introduced via an intravenous injection or feeding system, or otherwise internalized as is appropriate and directed.

The nutraceutical composition of the present invention comprises one or more of a processed Morinda citrifolia product present in an amount by weight between about 0.01 and 100 percent by weight, and preferably between 0.01 and 95 percent by weight. Several exemplary embodiments of formulations are provided below. However, these are only intended to be exemplary, as one ordinarily skilled in the art will recognize other formulations or compositions comprising the processed Morinda citrifolia product.

The processed Morinda citrifolia product is the active ingredient or contains one or more active ingredients, such as quercetin, rutin, scopoletin, octoanoic acid, potassium, vitamin C, terpenoids, alkaloids, anthraquinones (such as nordamnacanthal, morindone, rubiandin, B-sitosterol, carotene, vitamin A, flavone glycosides, linoleic acid, Alizarin, amino acides, acubin, L-asperuloside, caproic acid, caprylic acid, ursolic acid, and a putative proxeronine and others, for treating and relieving existing diabetes, as well as reducing the potential of developing diabetes in the future. Active ingredients may be extracted utilizing aqueous or organic solvents including various alcohol or alcohol-based solutions, such as methanol, ethanol, and ethyl acetate, and other alcohol-based derivatives using any known process in the art. The active ingredients of quercetin and rutin are present in amounts by weight ranging from 0.01-10 percent of the total formulation or composition. These amounts may be concentrated as well into a more potent concentration in which they are present in amounts ranging from 10 to 100 percent.

The nutraceutical composition comprising Morinda citrifolia may be prepared using any known means in the art. In addition, since the nutraceutical composition will most likely be consumed orally, it may contain one or more agents selected from the group consisting of sweetening agents, flavoring agents, coloring agents, preserving agents, and other medicinal agents as directed.

The present invention further features a method of administering a nutraceutical composition comprising one or more processed Morinda citrifolia products to affect cholesterol levels, lower LDL cholesterol levels and to inhibit HMG-CoA Reductase by providing a nutraceutical composition or treatment formulated. The method for administering a nutraceutical, or the method for treating a diabetes, comprises the steps of (a) formulating a nutraceutical composition comprising in part a processed Morinda citrifolia product present in an amount between about 0.01 and 95 percent by weight, wherein the composition also comprises a carrier, such as water or purified water, and other natural or artificial ingredients; (b) introducing the nutraceutical composition into the body, such that the processed Morinda citrifolia product is sufficiently internalized; (c) repeating the above steps as often as necessary to provide an effective amount of the processed Morinda citrifolia product to the body of the patient to positively affect cholesterol levels, lower LDL cholesterol levels, to inhibit HMG-CoA Reductase and/or decrease the absorption of fatty acids across the intestinal epithelium.

The step of introducing the nutraceutical composition into the body comprises one of ingesting the composition orally. Ingesting the nutraceutical orally means the nutraceutical composition may be formulated as a liquid, gel, solid, or some other type that would allow the composition to be quickly digested and concentrated within the body. It is important to note that the step of administering the nutraceutical composition should be carried out in an effective manner so that the greatest concentration of nutraceutical composition, and particularly the processed Morinda citrifolia product, is internalized and absorbed into the patient's body. In one embodiment, the nutraceutical composition is administered by taking between 1 teaspoon and 2 oz., and preferably 2 oz., of the nutraceutical composition every two hours each day, or at least twice a day. In addition, the nutraceutical composition is to be taken on an empty stomach, meaning at a period of time at least two hours prior to consumption of any food or drink. Following this, the nutraceutical composition is sufficiently allowed to absorb into the tissues of the body. Of course, one ordinarily skilled in the art will recognize that the amount of composition and frequency of use may vary from individual to individual. For example, the invention contemplates the administration of up to 10 ozs. for each administration.

In another method of the present invention, a person suffering from excess cholesterol levels takes at least one (1) ounce of Formulation One in the morning on an empty stomach, and at least one (1) ounce at night on an empty stomach, just prior to retiring to bed. In another method of the present invention, a person diagnosed with or experiencing excess cholesterol takes at least one ounce of Formulation Two twice a day. In addition, the step of administering the nutraceutical composition may include injecting the composition into the body using an intravenous pump.

The following compositions or formulations represent some of the preferred embodiments contemplated by the present invention.

| | |
|---|---|
| Ingredients | Percent by Weight |

Formulation One

| | |
|---|---|
| Morinda Citrifolia fruit juice | 100% |

Formulation Two

| | |
|---|---|
| Morinda Citrifolia fruit juice | 85-99.99% |
| Water | 0.1-15% |

Formulation Three

| | |
|---|---|
| Morinda Citrifolia fruit juice | 85-99.99% |
| Other fruit juices | 0.1-15% |

Formulation Four

| | |
|---|---|
| Morinda Citrifolia fruit juice | 50-90% |
| Water | 0.1-50% |
| Other fruit juices | 0.1-30% |

Formulation Five

| | |
|---|---|
| Morinda Citrifolia extract | 100% |

Formulation Six

| | |
|---|---|
| Morinda Citrifolia extract | 50-90% |
| Water | 0.1-50% |

Formulation Seven

| | |
|---|---|
| Morinda Citrifolia extract | 50-90% |
| Other fruit juices | 0.1-30% |

Formulation Eight

| | |
|---|---|
| Morinda Citrifolia extract | 50-90% |
| Water | 0.1-50% |
| Other fruit juices | 0.1-30% |

Formulation Nine

| | |
|---|---|
| Morinda Citrifolia extract | 0.1-50% |
| Water | 0.1-50% |

3. Positive Effect on Lipoprotein Levels

When evaluated on a Quantimetrix Lipoprint System LDL Subfraction device a typical lipoprint profile consists of one VLDL band, three mid-bands, up to seven LDL bands, and one HDL band by an electrophoresis assay. Large LDL is predominant in individuals with phenotype A while individuals in phenotype B have a predominance of small LDL. Phenotype A is the most common, whereas phenotype B often coexists with other lipoprotein abnormalities, notably elevated plasma triglycerides and low HDL cholesterol. Several case-control retrospective surveys suggest that the more abnormal phenotype B confers an increased risk for coronary diseases and large prospective studies consistently show that phenotype B is associated with an increased risk of coronary artery disease.

Studies have been conducted in support of this invention that indicate that the administration of nutraceutical comprising Morinda citrifolia, processed according to this invention, has a heart protective effect by improving lipoprotein profiles. See Example 1. Specifically, the administration of the composition of this invention lowers total cholesterol levels, lowers LDL levels, and increase HDL levels. Notably, administration of the nutraceutical disclosed in this invention is able to selectively decrease the deleterious portions of LDLs that may be a risk marker for cardiac disease in smokers. Smoking-specific DNA adducts and other biomarkers in current smokers may also be affected. By modifying the phenotype of their lipoprotein profile and decreasing the deleterious portions of LDL and increasing HDL, the nutraceutical claimed in this invention provides significant prophylactic benefits. Consequently, embodiments of the invention have wide application, including selectively decreasing LDLs in smokers, as LDLs may be a risk marker for heart attacks in smokers.

4. HMG-CoA Reductase Inhibition

Before cholesterol-lowering compounds and drugs are used in the market, they are typically tested first against the HMG-CoA Reductase enzyme, then in mice, and then in patients who suffer from high cholesterol levels. As illustrated by Examples 2 and 3 herein, embodiments of the present invention have been tested against the HMG-CoA Reductase enzyme. Specifically, Examples 2 and 3 illustrate the results of two in-vitro studies that confirmed that concentrates of processed Morinda citrifolia products (“TNJ” being an evaporative concentrate, and “TNCONC” being a freeze concentrate) could have a positive effect on cholesterol levels in-vivo. The percentage of concentration refers to the concentration strength of the particular concentrate tested; that is, the strength of concentration relative to the processed Morinda citrifolia product from which the concentrate was obtained. It will be noted that, while the in-vivo studies have not yet been performed, these might be performed by subjecting mice bred for high cholesterol levels to various concentrations of TNJ and TNCONC. Further, the invention contemplates utilizing studies in which the nutraceutical of the invention is administered to patients who suffer from high cholesterol levels.

It can be seen in the Examples that the 1% TNCONC has virtually the same potency as the 10% TNJ. It is expected that the compositions having Morinda citrifolia fruit juice concentrate obtained by freeze concentration will be more effective in inhibiting HMG-CoA Reductase because the proprietary process of freeze concentration ensures that the TNCONC is more potent than TNJ. Unlike the evaporative concentration process used with TNJ, the freeze concentration process used with TNCONC does not involve the use of heat; therefore, the volatiles and other natural compounds present in the Morinda citrifolia fruit are preserved in great abundance. The present invention contemplates incorporating TNCONC (or a bioactive fraction of it) into a drug form or other natural specialty product that can be used specifically for lowering cholesterol levels and/or to treat specific ailments or diseases.

5. Lipase Inhibition

Most lipids are ingested in the form of triacylglycerols, but must be degraded to fatty acids for absorption across the intestinal epithelium. Lipases digest the triacylglycerols into free fatty acids and monoacylglycerol. Gastric lipase, secreted by the stomach lining, has a pH value for optimal activity around neutrality and would appear, therefore, to be essentially inactive in the strongly acid environment of the stomach. It is suggested that this enzyme is more important for infant digestion since the gastric pH in infancy is much less acid than later in life. Most lipid digestion in the adult occurs in the upper loop of the small intestine and is accomplished by a lipase secreted by the pancreas. These digestion products are carried in micelles to the intestinal epithelium where they are absorbed across the plasma membrane. Inhibition of lipases responsible for the degradation of tracylglycerols would decrease the absorption of fatty acids across the intestinal epithelium. Reducing absorption of fatty acids has been found to lower blood cholesterol levels. See Example 4.

Unless otherwise indicated, any numbers expressing quantities of ingredients, reaction conditions, and so forth present in the specification or any claims or drawings are to be understood as being modified in all instances by the term “about.” Accordingly, unless indicated to the contrary, the numerical parameters set forth herein are approximations that may vary depending upon the desired properties sought to be obtained by the present invention. At the very least, each numerical parameter should at least be construed in light of the number of reported significant digits and by applying ordinary rounding techniques.

Notwithstanding that any numerical ranges and parameters that set forth the broad scope of the invention are approximations, the numerical values set forth in the specific examples are reported as precisely as possible. Any numerical value, however, inherently contains certain errors necessarily resulting from the standard deviation found in their respective testing measurements.

While illustrative embodiments of the invention have been described herein, the present invention is not limited to the various preferred embodiments described herein, but includes any and all embodiments having modifications, omissions, combinations, adaptations, and/or alterations as would be appreciated by those in the art based on the present disclosure. The limitations in any claims are to be interpreted broadly based on the language employed in the claims and not limited to examples described herein, which examples are to be construed as non-exclusive. For example, in the present disclosure, the term “preferably” should be construed as meaning “preferably, but not limited to.” The present invention may be embodied in other specific forms without departing from its spirit or essential characteristics. The described embodiments are to be considered in all respects only as illustrative and not restrictive.

### EXAMPLES

Studies were conducted, which demonstrate that administration of the nutraceutical disclosed herein improves lipoprotein profiles, reducing LDL levels, increases HDL levels, inhibit lipases which may decrease the absorption of fatty acids across the intestinal epithelium and inhibits HMG-CoA Reductase in living organisms utilizing Morinda citrifolia L.

### Example 1

A study was conducted to determine whether Morinda citrifolia fruit juice was able to improve the profiles of lipoproteins. A Quantimetrix Lipoprint System LDL Subfraction device was used to measure lipoprotein cholesterol for lipoprotein fractions and subfractions from VLDL to LDL in plasma. A randomized, double blind, placebo-controlled clinical trial with Morinda citrifolia fruit juice was conducted for one month. The subjects were supplemented twice daily with two ounces of Morinda citrifolia fruit juice, specifically TAHITIAN NONI® brand fruit juice and a placebo twice a day for 30 days. The total blood cholesterol, VLDL, LDL, HDL, and a profile of lipoproteins were determined before and after the trial. There was no effect on the placebo group on the total cholesterol VLDL, HDL, and LDL subfractions. Total cholesterol, total LDL in the TAHITIAN NONI® juice group was decreased by 6 percent, respectively, after a one-month clinical trial. HDL in the TAHITIAN NONI® juice group was increased up to 16 percent. Subfractions three and four of LDL were decreased by 30 percent and 57 percent, respectively. The profiles of lipoprotein in the TAHITIAN NONI® group were shifted (their particle size was increased) and phenotypes of lipoprotein were changed in the TAHITIAN NONI® group. Twenty-five (25) percent of the original type B phenotype individuals were changed to type A. Twenty-five (25) percent of phenotype B was changed to I, and 50 percent of type I was changed to type A. The ratio of cholesterol/HDL was decreased by 18 percent, and the ratio of LDL/HDL was decreased by 22 percent. These results indicate that TAHITIAN NONI® juice might have a heart protective effect by improving lipoprotein profiles and lower total cholesterol, LDL, and increase HDL. Notably, TAHITIAN NONI® juice is able to selectively decrease the deleterious portions of “subfractions 3 and 4” of LDL that may be a risk marker for cardiac disease in smokers. Smoking-specific DNA adducts and other biomarkers in current smokers may also be affected. By modifying the phenotype of their lipoprotein profile and decreasing the deleterious portions of LDL and increasing HDL, TAHITIAN NONI® juice provides significant prophylactic benefits.

### Example 2

Research was performed to evaluate, in Enzyme assays, the activity of the processed Morinda citrifolia products. In the research performed a 10% Concentration of TNJ inhibited HMG-CoA Reductase activities by 57%, wherein “TNJ” is an evaporative concentrate of Morinda citrifolia juice. Methods employed in this study have been adapted from the scientific literature to maximize reliability and reproducibility. Reference standards were run as an integral part of each assay to ensure the validity of the results obtained. Biochemical assay results are presented as the percent inhibition of specific binding or activity. Significant responses are 50% inhibition or stimulation for Biochemical assays. For primary assays, only the lowest concentration with a significant response judged by the assay criteria. Where applicable, either the secondary assay results with the lowest dose/concentration meeting the significance criteria or, if inactive, the highest dose/concentration that did not meet the significance criteria is shown.

### Example 3

Research was performed to evaluate, in Enzyme assays, the activity of the processed Morinda citrifolia products. Methods employed in this study have been adapted from the scientific literature to maximize reliability and reproducibility. Reference standards were run as an integral part of each assay to ensure the validity of the results obtained. Biochemical assay results are presented as the percent inhibition of specific binding or activity. Significant responses are 50% inhibition or stimulation for Biochemical assays. For primary assays, only the lowest concentration with a significant response judged by the assay criteria. Where applicable, either the secondary assay results with the lowest dose/concentration meeting the significance criteria or, if inactive, the highest dose/concentration that did not meet the significance criteria is shown. Processed Morinda citrifolia products utilized in the assays included “TNCONC” which is a freeze concentrate of Morinda citrifolia juice processed according to the present invention. In the assays performed a 1% solution of TNCONC inhibited HMG-CoA Reductase activities by 58%, a 5% solution of TNCONC inhibited HMG-CoA Reductase activities by 94%, and a 10% TNCONC solution inhibited HMG-CoA Reductase activities by 96%.

### Example 4

Research was performed to evaluate, in in-vitro Enzyme assays, the activity of processed Morinda citrifolia products. Methods employed in this study have been adapted from the scientific literature to maximize reliability and reproducibility. Reference standards were run as an integral part of each assay to ensure the validity of the results obtained. Biochemical assay results are presented as the percent inhibition of specific binding or activity. Significant responses are 50% inhibition or stimulation for Biochemical assays. For primary assays, only the lowest concentration with a significant response judged by the assay criteria. Where applicable, either the secondary assay results with the lowest dose/concentration meeting the significance criteria or, if inactive, the highest dose/concentration that did not meet the significance criteria is shown. Processed Morinda citrifolia products include “TNCMP1” being an evaporative concentrate. In the assays performed a 1% TNCMP1 solution inhibited lipase enzyme activity by 11% inhibition, and a 5% TNCMP1 solution inhibited lipase enzyme activity by 83%.

1. A formulation adapted for improving lipoprotein profiles in mammals comprising: at least one processed Morinda citrifolia product present in an amount by weight between about 0.1 and 99 percent.

2. The formulation of claim 1, wherein said Morinda citrifolia product is used with a carrier medium.

3. The formulation of claim 1, wherein said processed Morinda citrifolia product comprises a processed Morinda citrifolia selected from a group consisting of: extract from the leaves of Morinda citrifolia, leaf hot water extract present in an amount by weight between about 0.1 and 50 percent, processed Morinda citrifolia leaf ethanol extract present in an amount by weight between about 0.1 and 50 percent, processed Morinda citrifolia leaf steam distillation extract present in an amount by weight between about 0.1 and 50 percent, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice.

4. The formulation of claim 1, comprising at least one active ingredient selected from a group consisting of quercetin, rutin, scopoletin, octoanoic acid, potassium, vitamin C, terpenoids, alkaloids, anthraquinones, nordamnacanthal, morindone, rubiandin, B-sitosterol, carotene, vitamin A, flavone glycosides, linoleic acid, Alizarin, amino acids, acubin, L-asperuloside, caproic acid, caprylic acid, ursolic acid, and putative proxeronines.

5. The formulation of claim 1, wherein said formulation is administered to a patient by at least one method selected from a list consisting of orally, intravenously, and systemically.

6. The formulation of claim 1, further comprising at least one other ingredient selected from the group consisting of processed Morinda citrifolia products, food supplements, dietary supplements, other fruit juices, other natural ingredients, natural flavorings, artificial flavorings, natural sweeteners, artificial sweeteners, natural coloring, and artificial coloring.

7. A formulation comprising:
at least one processed Morinda citrifolia product present in an amount by weight between about 0.1 and 99 percent, wherein the formulation is adapted to affect mammals in a way selected from a group consisting of: inhibit HMG-CoA Reductase, reduce low-density lipoprotein levels, increase high-density lipoprotein levels, protect by prophylactic treatment from tobacco smoke-induced cardiac disease, and decrease the absorption of fatty acids across the intestinal epithelium.

8. A method for improving lipoprotein profiles in mammals comprising the step of:
administering a formulation containing at least one processed Morinda citrifolia product present in an amount by weight between about 0.1 and 99 percent.

9. The method of claim 8, wherein two ounces of the formulation is administered twice daily.

10. The method of claim 8, wherein said Morinda citrifolia product is administered with a carrier medium.

11. The method of claim 8, wherein said processed Morinda citrifolia product comprises a processed Morinda citrifolia selected from a group consisting of: extract from the leaves of Morinda citrifolia, leaf hot water extract present in an amount by weight between about 0.1 and 50 percent, processed Morinda citrifolia leaf ethanol extract present in an amount by weight between about 0.1 and 50 percent, processed Morinda citrifolia leaf steam distillation extract present in an amount by weight between about 0.1 and 50 percent, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice.

12. The method of claim 8, wherein the formulation comprises at least one active ingredient selected from a group consisting of quercetin, rutin, scopoletin, octoanoic acid, potassium, vitamin C, terpenoids, alkaloids, anthraquinones, nordamnacanthal, morindone, rubiandin, B-sitosterol, carotene, vitamin A, flavone glycosides, linoleic acid, Alizarin, amino acids, acubin, L-asperuloside, caproic acid, caprylic acid, ursolic acid, and putative proxeronines.

13. The method of claim 8, wherein the formulation further comprising at least one other ingredient selected from the group consisting of processed Morinda citrifolia products, food supplements, dietary supplements, other fruit juices, other natural ingredients, natural flavorings, artificial flavorings, natural sweeteners, artificial sweeteners, natural coloring, and artificial coloring.

14. The method of claim 8, further comprising the step of concurrently administering said formulation with another medication designed to improve lipoprotein profiles and its associated conditions, wherein said formulation increases the efficacy of said medication.

15. The method of claim 8, wherein said formulation is administered in an amount between about 1 teaspoon and 2 ounces at least twice daily on an empty stomach each day.

16. A method of treating mammals comprising:
administering a formulation containing at least one processed Morinda citrifolia product present in an amount by weight between about 0.1 and 99 percent, wherein the formulation is adapted to affect mammals in a way selected from a group consisting of: inhibit HMG-CoA Reductase, reduce low-density lipoprotein levels, increase high-density lipoprotein levels, to protect by prophylactic treatment from tobacco smoke-induced cardiac disease, and decrease the absorption of fatty acids across the intestinal epithelium.
